(19) United States
(12) Patent Application Publication
(10) Pub. No.: US 2026/0167670 A1
(43) Pub. Date: Jun. 18, 2026

(54) METHOD FOR SCREENING AND PREPARING ANTIOXIDANT PEPTIDE DERIVED FROM MARE'S MILK WHEY PROTEIN AND USE THEREOF

(71) Applicant: China Agricultural University, Beijing (CN)

(72) Inventors: Xiaoyun He, Beijing (CN); Lifang Yan, Beijing (CN); Qin Wang, Beijing (CN); Yue Luan, Beijing (CN); Yi Li, Beijing (CN); Yang Pang, Beijing (CN)

(21) Appl. No.: 19/395,945

(22) Filed: Nov. 20, 2025

(30) Foreign Application Priority Data
Nov. 20, 2024 (CN) 202411657823.1

(51) Int. Cl. C07K 7/06; A61K 38/00; A61P 39/06

(52) U.S. Cl. CPC C07K 7/06; A61P 39/06; A61K 38/00

(57) ABSTRACT

A method for screening and preparing antioxidant peptides from mare's milk whey protein and uses thereof. Potential antioxidant polypeptides are screened based on in silico enzymolysis and computer-based prediction, followed by molecular docking with the keap1 protein. The feasibility of obtaining antioxidant peptides is then validated by simulating gastrointestinal digestion using pepsin, trypsin, and chymotrypsin. Subsequently, high-purity antioxidant peptides are chemically synthesized and subjected to in vitro chemical antioxidant activity assays.

### CROSS REFERENCE TO RELATED APPLICATION

This patent application claims the benefit and priority of Chinese Patent Application No. 202411657823.1 filed with the China National Intellectual Property Administration on Nov. 20, 2024, the disclosure of which is incorporated by reference herein in its entirety as part of the present application.

### INCORPORATION BY REFERENCE OF SEQUENCE LISTING

A computer readable XML file entitled “Sequence Listing”, that was created on Nov. 18, 2025, with a file size of 14,357 bytes, contains the sequence listing for this application, has been filed with this application, and is hereby incorporated by reference in its entirety.

### TECHNICAL FIELD

The present disclosure belongs to the field of polypeptide products and molecular biology technology, and in particular relates to a method for screening and preparing an antioxidant peptide derived from mare's milk whey protein and use thereof.

### BACKGROUND

Oxidation is an inevitable process in all organisms. During oxidative metabolism, free radicals and reactive oxygen species (ROS) are generated. An imbalance between the generation of these free radicals and the body's antioxidant defenses leads to dysfunction of antioxidant system, resulting in harmful and irreversible chemical modifications such as cell death, apoptosis, lipid oxidation, and degradation of cellular components, ultimately contributing to various human diseases, including cancer, inflammation, cardiovascular disease, and neurodegenerative disease. In another aspect, the oxidation of food during storage leads to the loss of nutrients, spoilage and off-flavor formation, resulting in quality deterioration. Therefore, it is necessary to develop more antioxidants to scavenge free radicals and prevent food oxidation. Compared with natural antioxidants, synthetic antioxidants pose potential risks when used. Consequently, there is a need to develop natural antioxidants from food sources that may replace synthetic antioxidants with side effects as anti-oxidative food additives.

Antioxidant peptides are natural antioxidants, which upon entering the human body, inhibit the formation of free radicals and scavenge existing free radicals and reactive oxygen species. Studies have shown that mare's milk is highly nutritious and, due to its similar composition and low allergen content, are used as a substitute for human milk. It has been found to contain various peptides capable of preventing diabetes, lowering blood pressure, and enhancing immune function. However, research on its antioxidant peptides remains limited. This study screened peptides with high antioxidant activity from mare's milk whey protein via an in silico screening method, indicating that the hydrolysates of mare's milk whey protein have potential antioxidant bioactivity. Currently, methods such as ABTS and DPPH radical scavenging assays are commonly used in vitro to evaluate the efficiency of antioxidant peptides in different systems. However, verification of their antioxidant activity at the cellular level provides a more direct reflection of their value for application in organisms.

### SUMMARY

The object of the present disclosure is to provide an antioxidant peptide derived from mare's milk whey protein with antioxidant function, which exhibits good antioxidant activity both in vitro and at the cellular level, and is useful in the preparation of food, drugs, cosmetics and other products.

The object of the present disclosure is achieved by the following technical solution: An antioxidant peptide derived from mare's milk whey protein with antioxidant function, wherein the amino acid sequence of the antioxidant peptide is ALQPLPGR as set forth in SEQ ID NO: 4.

The present disclosure also provides: a method for screening and preparing the antioxidant peptide derived from mare's milk whey protein, wherein the method for screening the antioxidant peptide derived from mare's milk whey protein described above comprises the steps of:
(1) co-screening sequences of mare's milk whey protein based on relative abundance in the UniProt database;
In some embodiments, screening a total of two sequences of mare's milk whey protein according to the relative abundance in the UniProt database (www.uniprot.org/), with information regarding their entries, protein names, amino acid sequence lengths and molecular weights being displayed.
(2) performing in silico gastrointestinal digestion, and screening dipeptides, tripeptides, tetrapeptides, pentapeptides, and polypeptides from the hydrolysates generated with selected enzymes to form a peptide library;
In some embodiments, performing virtual gastrointestinal hydrolysis of all the screened proteins using the PeptideCutter program (www.expasy.org/resources/peptidecutter) in BIOPEP with the enzymes pepsin (at pH<2), trypsin, and chymotrypsin (high specificity), and subsequently, screening dipeptides, tripeptides, tetrapeptides, pentapeptides, and polypeptides from the resulting hydrolysates to form a peptide library.
(3) importing the polypeptide sequences from the peptide library into polypeptide bioactivity prediction websites for peptide segment activity prediction, and screening for novel peptides;
In some embodiments, predicting the bioactivity probability of each peptide segment in the peptide library using PeptideRanker (http://distilldeep.ucd.ie/PeptideRanker/), and screening peptide segments with a score greater than 0.5, wherein a peptide with a score greater than 0.5 is considered to have high potential for significant bioactivity; and screening novel peptides among the candidate peptide segments using the BIOPEP-UWM Bioactive Peptide Database (biochemia.uwm.edu.pl/en/biopep-uwm-2/).
(4) predicting the antioxidant activity of the novel peptides via a polypeptide antioxidant activity prediction website;
In some embodiments, predicting whether the novel peptides possess free radical-scavenging capacity using the AnOxPePred 1.0/prediction tool (services.healthtech.dtu.dk/services/).
(5) selecting higher-scoring polypeptide sequences and predicting the toxicity, isoelectric point, and hydrophilicity of the polypeptides using toxicity prediction tools;
In some embodiments, screening non-toxic, highly active polypeptides using the prediction tool from the toxicity prediction website Toixinpred (webs.iiitd.edu.in/raghava/toxinpred/index.html4); and predicting properties using ADMET (admetmesh.scbdd.com/), wherein the polypeptide properties include blood-brain barrier penetration, human intestinal absorption, carcinogenicity, and acute oral toxicity.
(6) performing molecular docking between the polypeptides screened above and the keap1 protein to identify active polypeptides with strong binding affinity;
In some embodiments, performing molecular docking between the finally screened polypeptides and the Keap1 protein; downloading the protein structure of the kelch domain of the Keap1 protein (PDB ID: 1U6D) from the PDB database (www.rcsb.org/), and modeling the peptide chains using the Maestro 11.0 software; performing molecular docking with the peptide chains as ligands and the Keap1 kelch domain as the receptor; using PyMOL software (version 4.3.0, pymol.org/) for dehydration and removal of organic matters, and using AutodockTools (http://mgltools.scripps.edu/downloads) for hydrogen addition and charge assignment; subjecting polypeptides to energy minimization, followed by protonating under neutral conditions (pH=7) using the H++3 online server; then assigning Amber14SB charges using the UCSF Chimera software; finally, selecting a conformation with a top docking score for subsequent analysis, performing 3D visualization using Schrödinger and Discovery Studio. The docking energy is generally a negative value; a smaller value indicates a higher docking score, meaning tighter binding between the ligand and the receptor protein, stronger interaction, and a greater potential for antioxidant activity.
(7) verifying whether the active polypeptide segments obtained through virtual screening can be actually obtained from mare's milk whey protein;
In some embodiments, centrifuging fresh mare's milk at 3000 g for 20 min to remove milk fat; adjusting skimmed mare's milk to pH 4.6 with acetic acid to precipitate casein; removing the casein by centrifugation at 12000 g for 20 min at 4° C.; collecting the resulting supernatant to obtain the whey protein, lyophilizing and storing for later use.

Weighing the whey protein and preparing as a solution (50 mg/ml), and ultrasonicating for 15 min to facilitate dissolution. The hydrolysis protocol is as follows: first adjusting the pH to 2.0 using 1 M HCL, followed by the addition of pepsin. Then raising the pH to 7.6 for the addition of trypsin, and finally adjusting to 8.0 for the addition of chymotrypsin. Each enzyme is added at a ratio of 6000 U/g of protein. The sequential hydrolysis is performed for 2 h each at 37° C. in a water bath under magnetic stirring. After hydrolysis is complete, inactivating the enzymes by heating at 95° C. for 15 min; cooling the hydrolysate to room temperature, adjusting to neutral pH, and centrifuging at 12000 g for 15 min at 4° C. Filtering the supernatant sequentially through 0.45 μm and 0.22 μm membranes; lyophilizing the filtrate to obtain a crude polypeptide powder, which was designated as M1. Then dissolving the crude polypeptide powder and subjecting to ultrafiltration using a 3 KDa member to separate the sample into two fractions: one with a molecular weights greater than 3 KDa (designated as M2) and the other less than 3 KDa (designated as M3), both fractions are subsequently lyophilized. Given that the molecular weights of the predicted antioxidant peptides are all below 1500 KDa, the fraction with a molecular weight <3 kDa (M3) is selected for further peptide identification. Additionally, the antioxidant activities of all three components (M1, M2, and M3) are evaluated.
(8) performing peptidomics analysis and detection on the enzymatic hydrolysates of the mare's milk whey protein to identify the sequence information of the peptide segments after enzymolysis;
In some embodiments, performing peptide segment sequence analysis using a Vanquish Neo/Orbitrap Exploris 480 liquid chromatography system-mass spectrometer.
(9) chemically synthesizing highly active polypeptide sequences to obtain the antioxidant peptide derived from mare's milk whey protein with a purity of 98% or more, and performing antioxidant activity verification.

The present disclosure also provides use of the method for screening and preparing the antioxidant peptide for the screening and preparation of the antioxidant peptide, specifically the use of the method for the screening and preparation of the antioxidant peptide derived from mare's milk whey protein.

Further, the present disclosure also provides use of the antioxidant peptide in food, drugs and cosmetics for defending against oxidative damage.

Compared with the prior art, the present disclosure has the following advantages:
1. A novel peptide with high antioxidant activity is obtained in the present disclosure through simulated hydrolysis and computer screening. By utilizing effective databases and molecular docking to simulate the primary target of antioxidant peptides, the rapid and efficient identification of antioxidant peptides is achieved. Compared with conventional production procedures for bioactive peptides, this bioinformatics technology has the advantages of time-saving, accuracy and high efficiency. Nevertheless, the practical efficacy of the predicted peptides remains a major concern. Therefore, conventional hydrolysis and characterization are conducted to validate the in silico approach and to experimentally confirm that the computationally screened candidate peptides are also present in the hydrolysates of mare's milk whey protein. These data indicate that two effective novel peptides are experimentally isolated from the hydrolysates.
2. The antioxidant peptides screened in the present disclosure exhibit robust and comprehensive antioxidant activity, and feature high efficiency, simple preparation method, and good reproducibility. They show broad prospects in the fields of food, pharmaceuticals, and cosmetics, providing an efficient pathway for the development of functional polypeptide products derived from mare's milk whey protein. The specific advantages are as follows:
(1) ALQPLPGR (SEQ ID NO: 4) has a scavenging rate comparable to GSH, showing excellent DPPH·free radical-scavenging activity and O⁻₂ superoxide anion scavenging activity, as well as a considerable scavenging capacity against ABTS·+ and ·OH free radicals, in addition to ferric ion reducing power;
(2) Results from cellular antioxidant activity assays indicate that the ALQPLPGR (SEQ ID NO: 4) peptide confers protection against H₂O₂-induced damage in HepG2 cells. Compared with the model group, the ALQPLPGR (SEQ ID NO: 4) peptide exhibits varying degrees of antioxidant activity in cells by significantly reducing malondialdehyde (MDA) production and intracellular reactive oxygen species (ROS) levels, while markedly enhancing the enzyme activities of superoxide dismutase (SOD), catalase (CAT), and glutathione peroxidase (GSH-Px). RT-PCR analysis further reveals that the peptide enhances the antioxidant capacity of HepG2 cells by activating the Nrf2 pathway.

### BRIEF DESCRIPTION OF THE DRAWINGS

FIG. 1 shows the three-dimensional (3D) diagram of the molecular docking of the peptide ALQPLPGR (SEQ ID NO: 4) with the Keap-1 protein.

FIG. 2 shows the three-dimensional (3D) perspective view of the molecular docking of the peptide ALQPLPGR (SEQ ID NO: 4) with the Keap-1 protein.

FIG. 3 shows the two-dimensional (2D) diagram of the molecular docking of the peptide ALQPLPGR (SEQ ID NO: 4) with the Keap-1 protein.

FIG. 4 shows the detailed local plan view of the molecular docking of the peptide ALQPLPGR (SEQ ID NO: 4) with the Keap-1 protein.

FIG. 5 shows the total ion chromatogram from the HPLC-MS/MS analysis of the peptide segments in the M3 component.

FIG. 6 shows the tandem mass spectrometry spectrum of the peptide ALQPLPGR (SEQ ID NO: 4) identified in the M3 component.

FIG. 7 shows the molecular weight distribution of the peptide segments in the M3 component.

FIG. 8 shows the composition of amino acids in the M3 component.

FIG. 9 shows the results of DPPH scavenging rate of the components with various molecular weights in the hydrolysates of mare's milk whey protein.

FIG. 10A shows the HPLC chromatogram of the polypeptide ALQPLPGR (SEQ ID NO: 4) after the chemical solid-phase synthesis.

FIG. 10B shows the MS spectrum of the polypeptide ALQPLPGR (SEQ ID NO: 4) after the chemical solid-phase synthesis.

FIG. 11 shows the DPPH·scavenging rate of the peptides at different concentrations.

FIG. 12 shows the ABTS·+scavenging rate of the peptides at different concentrations.

FIG. 13 shows the O⁻₂ superoxide anion scavenging rate of the peptides at different concentrations.

FIG. 14 shows the effect of H₂O₂ at different concentrations on the survival rate of HepG2 cells. Note: The one marked **** indicates very significant difference (P<0.0001).

FIG. 15 shows the effect of the peptides on the survival rate of HepG2 cells.

FIG. 16 shows the protective effect of the peptides against H₂O₂-induced oxidative damage in HepG2 cells. Note: 100 indicates 100 μg/ml peptide solution+H₂O₂; 200 indicates 200 μg/ml peptide solution+H₂O₂. * indicates a significant difference (P<0.05) for the control group compared to the model group; ###indicates a significant difference (P<0.001) and ####indicates a significant difference (P<0.0001) for the sample groups compared to the model group.

FIG. 17 shows the effects of the peptides on the MDA levels in HepG2 cells; Note: 200 indicates 200 μg/ml peptide solution+H₂O₂; * indicates a significant difference (P<0.05) for the control group compared to the model group; ##indicates a significant difference (P<0.01) for the sample group compared to the model group.

FIG. 18 shows the effects of the peptides on the SOD activity in HepG2 cells. Note: 200 indicates 200 μg/ml peptide solution+H₂O₂; ** indicates a significant difference (P<0.01) for the control group compared to the model group; ####indicates a significant difference (P<0.0001) for the sample group compared to the model group.

FIG. 19 shows the effects of the peptides on the CAT activity in HepG2 cells. Note: 200 indicates 200 μg/ml peptide solution+H₂O₂; **** indicates a significant difference (P<0.0001) for the control group compared to the model group; ##indicates a significant difference (P<0.01) for the sample group compared to the model group.

FIG. 20 shows the effects of the peptides on the GSH-Px activity in HepG2 cells. Note: 200 indicates 200 g/ml peptide solution+H₂O₂; * indicates a significant difference (P<0.05) for the control group compared to the model group; ###indicates a significant difference (P<0.001) for the sample group compared to the model group.

FIG. 21 shows the effects of the peptides on the expression of Keap-1/Nrf2 pathway-related genes in HepG2 cells. Note: * indicates a significant difference of P<0.05, ** indicates a significant difference of P<0.01, *** indicates a significant difference of P<0.001, and * indicates a significant difference of P<0.0001.

FIGS. 22A-22B show the effects of the peptides on the ROS levels in HepG2 cells; FIG. 22A shows the diagram of the ROS fluorescence results of HepG2 cells; FIG. 22B shows the quantitative analysis diagram of ROS fluorescence in HepG2 cells.

### DETAILED DESCRIPTION OF THE EMBODIMENTS

The concept and technical effects of the present disclosure will now be further elucidated with reference to specific examples, in order to fully understand the object, features and effect of the present disclosure. Unless otherwise specified, the methods are all conventional methods. The materials, unless otherwise indicated, can all be available through public commercial approaches. The schematic examples of the present disclosure and their descriptions are intended to explain the present disclosure, but do not constitute an undue limitation to the present disclosure. It should be noted that, in absence of conflicts, the examples and the features in the examples in the present disclosure may be combined with each other.

### Example 1: Virtual Enzymolysis and Screening of Antioxidant Peptide Derived from Mare's Milk Whey Protein

### 1. Experimental Methods

(1) According to the relative abundance in the UniProt database (www.uniprot.org/), a total of two sequences of mare's milk whey protein (see Table 1) were screened, and information was provided regarding their entries, protein names, amino acid sequence lengths and molecular weights. Gastrointestinal digestion was performed in silico and virtual gastrointestinal hydrolysis was performed on all the screened proteins using the PeptideCutter program (www.expasy.org/resources/peptidecutter) in BIOPEP with the enzymes pepsin (at pH<2), trypsin, and chymotrypsin (high specificity). Dipeptides, tripeptides, tetrapeptides, pentapeptides, and polypeptides were screened from the hydrolysates produced with the three enzymes to form a peptide library.

(2) The bioactivity probability of each peptide segment in the peptide library was predicted using PeptideRanker (http://distilldeep.ucd.ie/PeptideRanker/), and peptide segments with a score greater than 0.5 were screened, wherein a peptide with a score greater than 0.5 was considered to have a high potential for significant bioactivity.

(3) The BIOPEP-UWM Bioactive Peptide Database (biochemia.uwm.edu.pl/en/biopep-uwm-2/) was used to screen for novel peptides among the candidate peptide segments described above.

(4) The novel peptides were further predicted for free radical-scavenging capacity using the AnOxPePred 1.0/prediction tool (services.healthtech.dtu.dk/services/).

(5) The toxicity of the above-screened novel peptides with free radical scavenging capacity was evaluated using the prediction tool from the toxicity prediction website Toixinpred (webs.iiitd.edu.in/raghava/toxinpred/index.html4), and their basic properties such as isoelectric point and hydrophilicity were evaluated.

(6) The ADMET tool (admetmesh.scbdd.com/) was used for predicting properties, including polypeptide properties such as blood-brain barrier penetration, human intestinal absorption, carcinogenicity, and acute oral toxicity.

(7) Molecular docking was performed between the finally screened polypeptides and the Keap1 protein. The protein structure of the kelch domain of the Keap1 protein (PDB ID: 1U6D) was downloaded from the PDB database (www.rcsb.org/), and the peptide chains were modeled using the Maestro 11.0 software. Molecular docking was performed with the peptide chains as ligands and the Keap1 Kelch domain as the receptor. PyMOL software (version 4.3.0, pymol.org/) was used for dehydration and removal of organic matters, and the AutodockTools (http://mgltools.scripps.edu/downloads) was used for hydrogen addition and charge assignment. The polypeptides were subjected to energy minimization, followed by protonation under neutral conditions (pH=7) using the H++3 online server. The Amber14SB charges were then assigned using the UCSF Chimera software. Finally, the conformation with the top docking score was selected for subsequent analysis, with 3D visualization performed using Schrödinger and Discovery Studio. The docking energy is generally a negative value; a smaller value indicates a higher docking score, meaning tighter binding between the ligand and the receptor protein, stronger interaction, and a greater potential for antioxidant activity.

### 2. Experimental Results

### (1) the Table Below Shows the Information of the Whey Protein Sequences Screened

TABLE 1
Mare's milk whey protein sequences

| | Protein | | Molecular | | |
|---|---|---|---|---|---|
| Entry | name | Length | mass | Sequence | No: |
| P07380.LA | Beta- | 180 | 20.1 | MKCLLLALGLSLMCGNQAT | SEQ ID |
| CB2_ | lactoglobulin- | | | DIPQTMQDLDLQEVAGRWH | NO: 1 |
| HORSE | 2 | | | SVAMVASDISLLDSESVPLR | |
| | | | | VYVEELRPTPEGNLEIILRE | |
| | | | | GANHACVERNIVAQKTEDPA | |
| | | | | VFTVNYQGERKISVLDTDY | |
| | | | | AHYMFFCVGPPLPSAEHGM | |
| | | | | VCQYLARTQKVDEEVMEKF | |
| | | | | SRALQPLPGRVQIVQDPSGG | |
| | | | | QERCGF | |
| P02758.LA | Beta- | 180 | 20.3 | MKCLLLALGLALMCGIQAT | SEQ ID |
| CB1_ | lactoglobulin- | | | NIPQTMQDLDLQEVAGKWH | NO: 2 |
| HORSE | 1 | | | SVAMAASDISLLDSESAPLR | |
| | | | | VYIEKLRPTPEDNLEIILREG | |
| | | | | ENKGCAEKKIFAEKTESPAE | |
| | | | | FKINYLDEDTVFALDTDYK | |
| | | | | NYLFLCMKNAATPGQSLVC | |
| | | | | QYLARTQMVDEEIMEKFRR | |
| | | | | ALQPLPGRVQIVPDLTRMAE | |
| | | | | RCRI | |

(2) the Table Below Shows the Peptide Segments with Scores Greater than 0.5 Screened from the Peptide Library

TABLE 2
Whey protein peptide segments with bioactivity
scores greater than 0.5

| Peptide segments | Score | No: |
|---|---|---|
| MF | 0.996643 | — |
| IF | 0.949173 | — |
| CR | 0.865233 | — |
| CGF | 0.618551 | — |
| CVGPPLPSA | 0.569984 | SEQ ID NO: 3 |
| EHGMVCQY | | |
| LL | 0.990232 | — |
| LR | 0.702872 | — |
| CMK | 0.820967 | — |
| ALQPLPGR | 0.769236 | SEQ ID NO: 4 |
| LQPLPGR | 0.679308 | SEQ ID NO: 5 |

MF, IF, LL, and LR were bioactive peptides that have been discovered by searching the Bioactive Peptide Database. To focus on the screening of novel active peptides, these four were excluded from the library, and subsequent activity prediction was performed on the remaining peptide segments.

### (3) the Table Below Shows the Free Radical-Scavenging Capacity of the Novel Peptides

TABLE 3
Free radical scavenging scores
of novel peptides

| Serial | Peptide | | |
|---|---|---|---|
| No. | segments | FRS score | No: |
| 1 | CVGPPLPSA | 0.48011959 | SEQ ID NO: 3 |
| | EHGMVCQY | | |
| 2 | ALQPLPGR | 0.46208909 | SEQ ID NO: 4 |
| 3 | LQPLPGR | 0.45589432 | SEQ ID NO: 5 |
| 4 | CGF | 0.44257522 | — |
| 5 | CR | 0.40624395 | — |
| 6 | CMK | 0.38420343 | — |

TABLE 4
Novel peptide chelation scores

| Serial | Peptide | | |
|---|---|---|---|
| No. | segments | CHEL score | No: |
| 1 | LQPLPGR | 0.27767465 | SEQ ID NO: 5 |
| 2 | ALQPLPGR | 0.26800862 | SEQ ID NO: 4 |
| 3 | CGF | 0.25253952 | — |
| 4 | CMK | 0.2486506 | — |
| 5 | CR | 0.24400081 | — |
| 6 | CVGPPLPSA | 0.21451199 | SEQ ID NO: 3 |
| | EHGMVCQY | | |

In summary, the tables showed that all the polypeptides described above may have free radical-scavenging activity. Among them, the polypeptide ALQPLPGR (SEQ ID NO: 4) had good free radical-scavenging activity overall. It is believed that it had great free radical scavenging potential. The toxicity of the above peptides was then evaluated to confirm that they have no biological toxicity.

### (4) the Table Below Shows the Prediction of the Toxicity and Other Basic Properties of the Polypeptides

TABLE 5
Noval peptide toxicity prediction and basic properties

| Peptide | Toxicity | | Steric | Molecular | | Isoelectric |
|---|---|---|---|---|---|---|
| sequences | prediction | Hydrophobicity | hindrance | weight | Charge | point |
| ALQPLPGR (SEQ | Non- | −0.14 | 0.54 | 851.13 | 1.00 | 10.11 |
| ID NO: 4) | Toxin | | | | | |
| LQPLPGR (SEQ | Non- | −0.20 | 0.55 | 780.04 | 1.00 | 10.11 |
| ID NO: 5) | Toxin | | | | | |
| CGF | Non- | 0.27 | 0.67 | 325.41 | 0.00 | 5.85 |
| | Toxin | | | | | |
| CMK | Non- | −0.27 | 0.69 | 380.55 | 1.00 | 8.57 |
| | Toxin | | | | | |
| CR | Non- | −0.86 | 0.65 | 277.35 | 1.00 | 8.60 |
| | Toxin | | | | | |
| CVGPPLPSA | Non- | 0.02 | 0.56 | 1788.33 | −0.50 | 5.25 |
| EHGMVCQY | Toxin | | | | | |
| (SEQ ID | | | | | | |
| NO: 3) | | | | | | |

### (5) the Table Below Shows the Molecular Docking Binding Energies Between the Screened Candidate Peptides and the Keap1 Protein

TABLE 6
Molecular docking binding energies between
novel peptides and Keap1 protein

| Serial | | Affinity |
|---|---|---|
| No. | Peptide segments | (kcal/mol) |
| 1 | ALQPLPGR (SEQ ID NO: 4) | −8.1 |
| 2 | LQPLPGR (SEQ ID NO: 5) | −7.6 |
| 3 | CGF | −7.3 |
| 4 | CVGPPLPSAEHGMVCQY | −7.2 |
| | (SEQ ID NO: 3) | |
| 5 | CR | −7.0 |
| 6 | CMK | −6.2 |

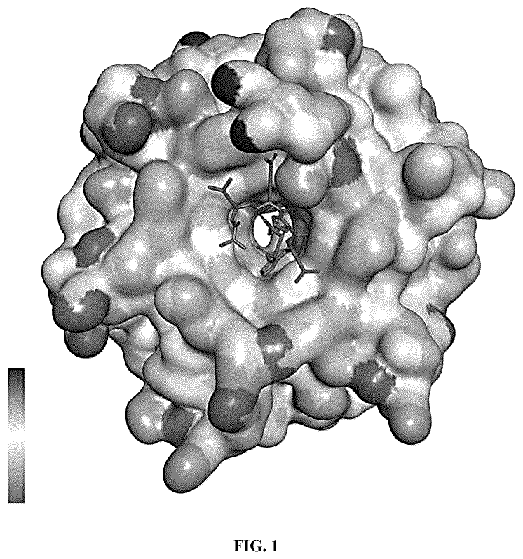

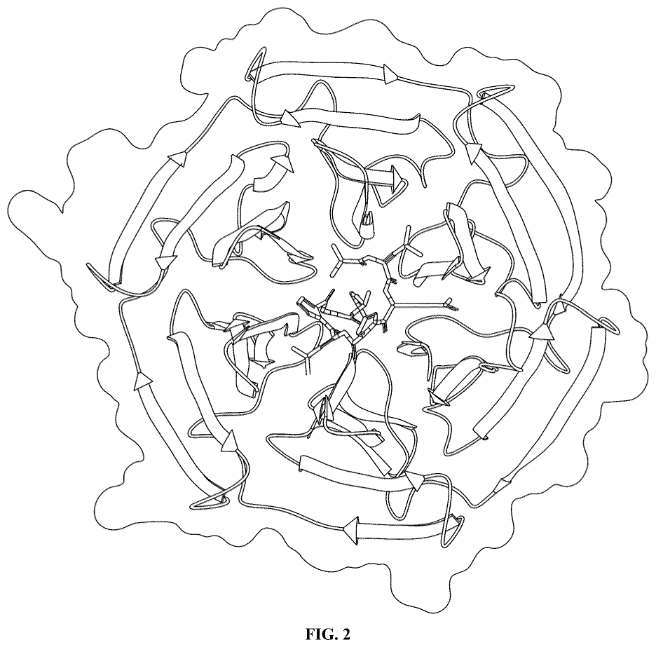

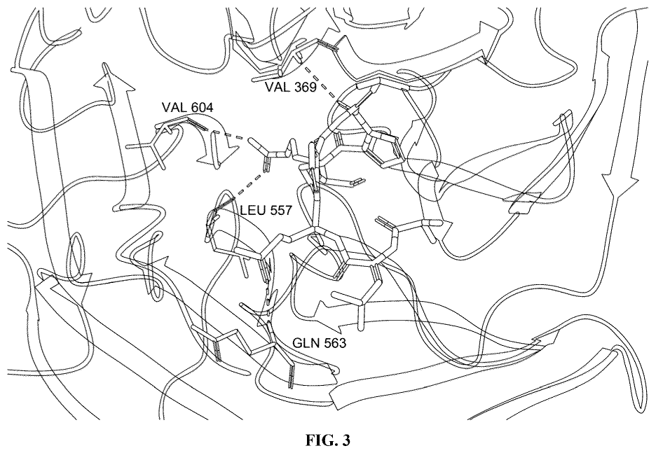

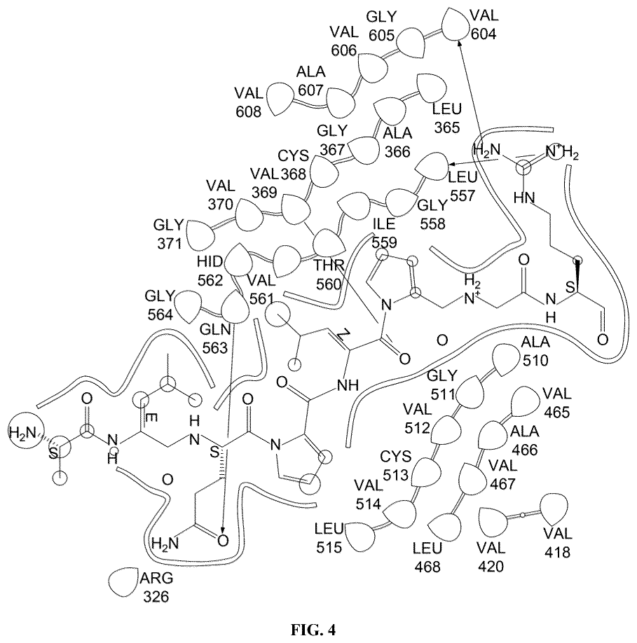

Table 6 showed the molecular docking binding energies between the six screened novel peptides and the Kelch domain of keap1. The molecular docking scores ranged from −6.2 kcal/mol (CMK) to −8.1 kcal/mol (ALQPLPGR,SEQ ID NO: 4). In other studies, the absolute value of the molecular docking score showed a positive correlation with the antioxidant activity, and a binding energy score <−8.0 kcal/mol was considered to indicate relatively strong binding force. With a score of −8.1 kcal/mol, ALQPLPGR (SEQ ID NO: 4) demonstrated tight binding to the Keap1 Kelch domain, indicating its strong potential as an antioxidant polypeptide. FIGS. 1-4 showed the diagrams of the molecular docking of the polypeptide ALQPLPGR (SEQ ID NO: 4) with the Kelch domain of Keap1, including 3D diagrams and detailed local view. It can be seen that the polypeptide ALQPLPGR (SEQ ID NO: 4) bound to the Kelch domain of keap1 via hydrogen bonds with VAL369, LEU557, GLN563, and VAL604, confirming it as our finally screened antioxidant peptide.

### Example 2: Preparation, Identification and Solid Phase Synthesis of Mare's Milk Whey Protein Antioxidant Peptides

### 1. Experimental Methods and Steps

(1) Preparation of Antioxidant Peptide from Mare's Milk Whey Protein

Fresh mare's milk was centrifuged at 3000 g for 20 min to remove milk fat. The skimmed mare's milk was adjusted to pH 4.6 with acetic acid to precipitate casein. The casein was subsequently removed by centrifugation at 12000 g for 20 min at 4° C. The resulting supernatant, i.e., whey protein, was collected, lyophilized, and stored for later use.

The whey protein was weighed and prepared as a solution (50 mg/ml) and ultrasonicated for 15 min to facilitate dissolution. The hydrolysis protocol was as follows: the pH was first adjusted to 2.0 using 1 M HCL, followed by the addition of pepsin; the pH was then raised to 7.6 for the addition of trypsin, and finally adjusted to 8.0 for the addition of chymotrypsin; each enzyme was added at a ratio of 6000 U/g of protein; the sequential hydrolysis was performed for 2 h each at 37° C. in a water bath under magnetic stirring. After hydrolysis was complete, the enzymes were inactivated by heating at 95° C. for 15 min. The hydrolysate was cooled to room temperature, adjusted to neutral pH, and centrifuged at 12000 g for 15 min at 4° C. The supernatant was sequentially filtered through 0.45 μm and 0.22 μm membranes. The filtrate was then lyophilized to obtain a crude polypeptide powder, designated as M1. The crude polypeptide powder was dissolved and subjected to ultrafiltration using a 3 KDa ultrafiltration tube to obtain two components with molecular weights >3 KDa (designated as M2) and <3 KDa (designated as M3), respectively, which were then lyophilized. Since the molecular weights of the predicted antioxidant peptides were all below 1500 KDa, the component with a molecular weight <3 kDa was utilized for peptide segment identification. Additionally, the antioxidant activities of all three components were determined.

### (2) Polypeptide Composition Analysis

### a) Pretreatment Method

Sample dissolution: 1 mg of the sample was weighed and dissolved in 0.1 mL of 50 mM NH₄HCO₃;

C₁₈ desalting (Stage-Tip): Activation: The desalting column was activated twice with 300 μL of 100% ACN. Equilibration: The desalting column was equilibrated twice with 200 μL of 0.1% TFA. Loading: The sample was loaded twice. Desalting: The column was desalted three times with 200 μL of 0.1% TFA. Elution: Peptides were eluted once with 300 μL of 80% ACN-20% 0.1% TFA, followed by vacuum drying at 45° C.

Peptide segment quantification: Exactly 25 μL of the sample was transferred to a new 1.5 mL EP tube using a pipette. Exactly 20 μL of the standard and the sample were transferred into the microplate wells using a pipette. 180 μL of working reagent was added to each well. The microplate was mixed on a microplate shaker for 30 s and then incubated at 37° C. for 15 min. The 96-well plate was cooled to room temperature and then placed in a full-wavelength microplate reader, and the absorbance at 480 nm was measured. A standard curve was plotted, and the sample concentration was calculated.

### b) Liquid Quality Detection

Vanquish Neo/Orbitrap Exploris 480 liquid chromatography system-mass spectrometer were used and the liquid chromatography conditions were:
1) Analytical column: 75 μm i.d.×25 cm, NanoViper C18 1.9 μm, 100 A
2) Mobile phase A: 0.1% FA;
3) Mobile phase B: 0.1% FA, 80% ACN;
4) Flow rate: 300 nL/min;
5) Analysis time per component: 66 min; and
6) Specific chromatographic conditions:

| Time (min) | Phase B |
|---|---|
| 0 | 4% |
| 2 | 8% |
| 35 | 28% |
| 55 | 40% |
| 56 | 95% |
| 66 | 95% |

Mass spectrometry full scan range: 100-1500 m/z; primary mass spectrometry resolution: 120000; AGC: Standard; Maximum IT: 20 ms; tandem mass spectrometry resolution: 15000; AGC: Standard; Maximum IT: 22 ms; cycle time: 1.3 s, peptide segment fragmentation collision energy: 30. The raw data of mass spectrometry was generated. The raw mass spectrometry files were searched against the target protein database using software with the following parameters:
1) Fixed modifications: Carbamidomethyl (C).
2) Variable modifications: Oxidation (M), Acetyl (Peptide N-term).
3) Enzyme: Non specific.
4) Database: uniprotkb_proteome_Equus_caballus_2024_06_24.
5) Primary mass spectrometry tolerance (Peptide Mass Tolerance): 20 ppm
6) Tandem mass spectrometry tolerance (Fragment Mass Tolerance): 0.02 Da

The fractions separated by chromatography continuously entered the mass spectrometer, which performed continuous scanning for data acquisition. Each scan produced one mass spectrum, ultimately yielding the total ion chromatogram and secondary spectra of different peptide segments.

c) To obtain the ALQPLPGR (SEQ ID NO: 4) polypeptide with higher purity for subsequent antioxidant activity detection, chemical solid-phase synthesis of the peptide was performed, and its purity was determined to be 98% or more by HPLC detection.

### 2. Experimental Results

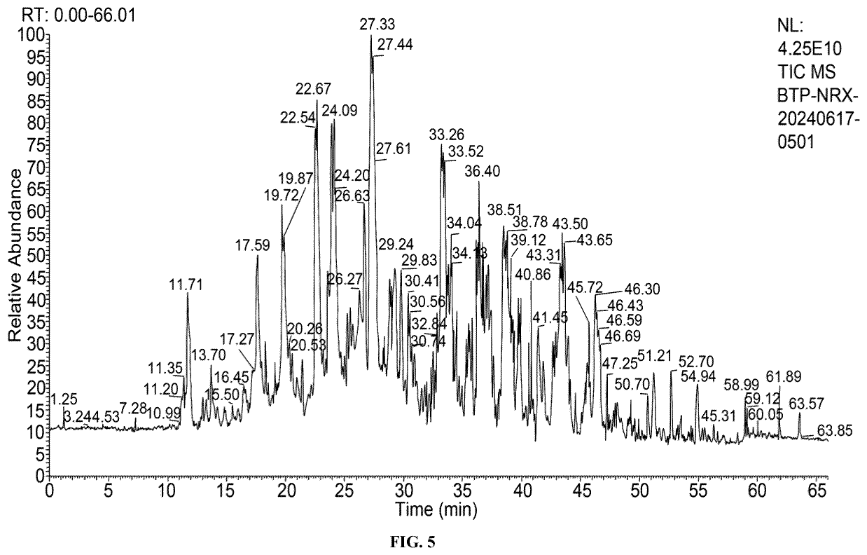

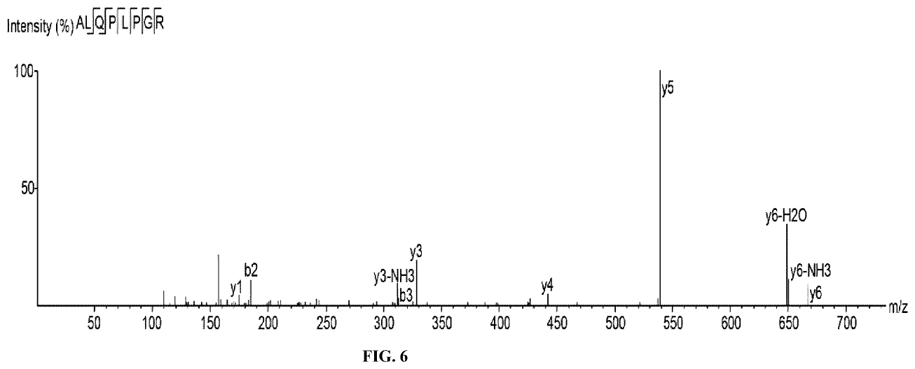

(1) The crude peptides obtained by simulated gastrointestinal digestion were detected by peptidomics, generating the HPLC-MS/MS total ion chromatogram in FIG. 5 and the tandem mass spectrum of ALQPLPGR (SEQ ID NO: 4) in FIG. 6. These results demonstrated that the polypeptide ALQPLPGR (SEQ ID NO: 4), which was identified through in silico screening, can be obtained from mare's milk whey protein via enzymatic hydrolysis.

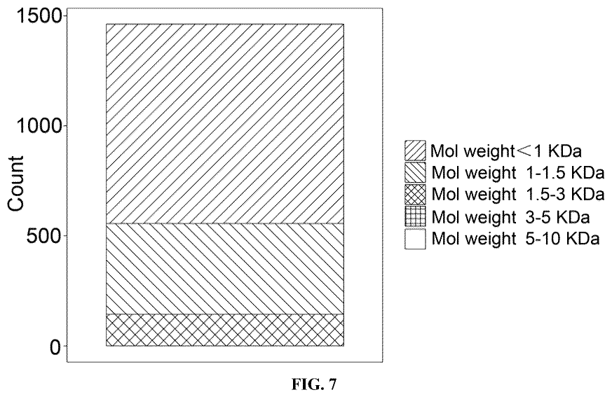

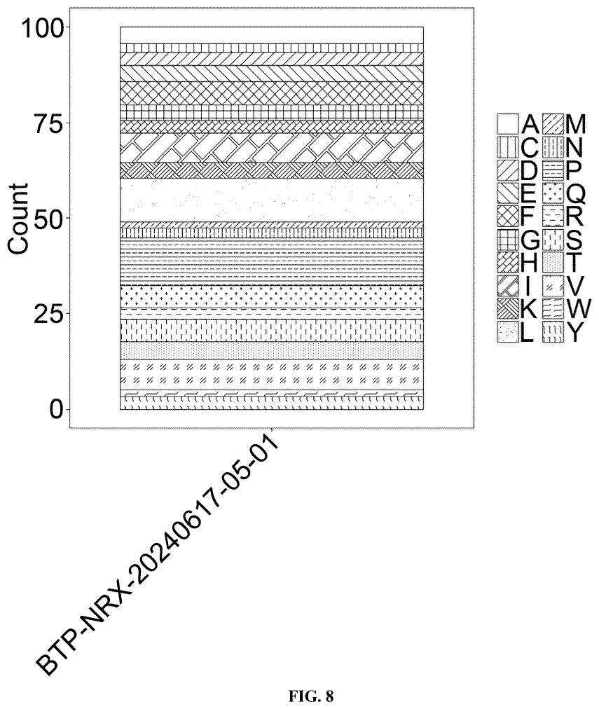

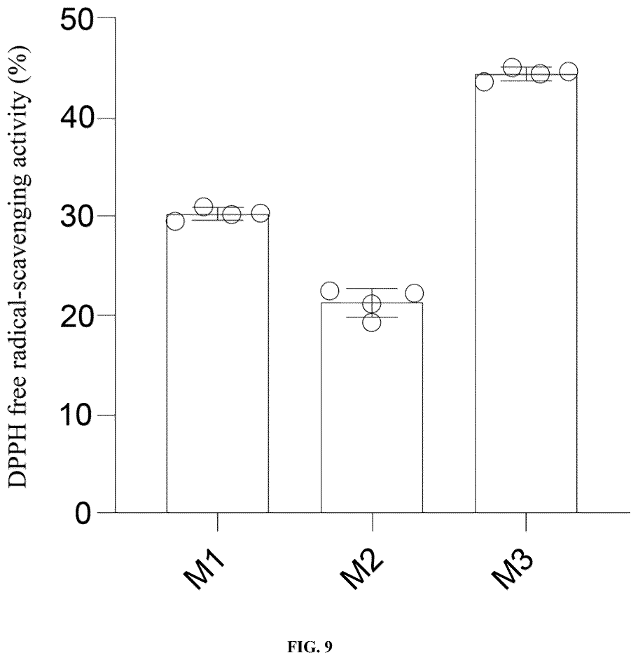

(2) Peptidomics analysis detected a total of 1463 polypeptides. FIG. 7 showed the molecular weight distribution of the peptide segments in the M3 component, revealing that all detected polypeptides were ≤3 KDa. This finding was consistent with the actual extraction situation. Among them, polypeptides with a molecular weight <1 KDa accounted for the largest proportion. Since shorter peptide segments may have higher antioxidant properties, it was speculated that the M3 component may have stronger antioxidant activity based on the molecular weight distribution. FIG. 8 showed the amino acid composition of the M3 component, among which leucine (L) and proline (P) were the most abundant. Both of them were hydrophobic amino acids. Hydrophobic amino acids stabilized free radicals by providing protons and were effective antioxidants. FIG. 9 showed the detected DPPH free radical-scavenging activity of the crude peptides extracted from mare's milk whey protein. The results indicated that the M3 component possessed high free radical-scavenging activity.

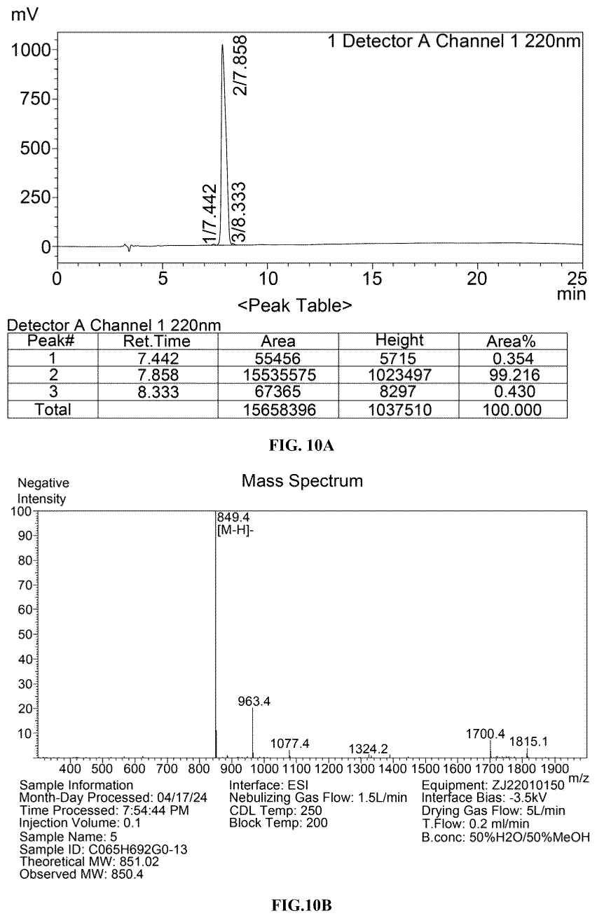

(3) FIG. 10A showed the HPLC chromatogram of the polypeptide ALQPLPGR (SEQ ID NO: 4) after chemical solid-phase synthesis, showing a purity of 99.216%; FIG. 10B showed the MS spectrum of the polypeptide ALQPLPGR (SEQ ID NO: 4) after the chemical solid-phase synthesis.

### Example 3: In Vitro Chemical Antioxidant Activity of Mare's Milk Whey Protein Antioxidant Peptide

### 1. Experimental Steps and Methods

### (1) Determination of DPPH·Scavenging Rate

2,2-diphenyl-1-picrylhydrazyl (DPPH) (19.7 mg) was weighed and dissolved in anhydrous ethanol and diluted to a final volume of 250 mL to prepare a 0.2 mmol/L DPPH solution. Peptide solutions at different concentrations were prepared. Experimental group As: 100 μL of peptide solution+100 μL of DPPH solution; Control group Ac: 100 μL of peptide solution+100 μL of anhydrous ethanol; Blank group Ab: 100 μL of distilled water+100 μL of DPPH solution. The reaction system was thoroughly mixed and left at room temperature for 30 min in the dark, and the absorbance at 517 nm was determined. The DPPH·scavenging rate was calculated according to Formula 1:

$$DPPH \cdot scavenging rate ( \% ) = ( 1 - \frac{As - Ac}{Ab} ) \times 100 \% \qquad ( 1 )$$

### (2) Determination of ABTS·+Scavenging Rate

The ABTS·+scavenging activity assay was carried out referring to the instructions of the Total Antioxidant Capacity Assay kit (Rapid ABTS method). The ABTS working reagent was prepared immediately before use, stored at room temperature protected in the dark, and used within 30 minutes. Peptide solutions at different concentrations were prepared. The antioxidant capacity of the antioxidant was expressed as the relative Trolox total antioxidant capacity. Therefore, Trolox was subjected to serial dilution to generate the standard curve. Experimental group: 20 μL of peroxidase working reagent+10 μL of peptide solution+170 μL of ABTS working reagent; standard curve group: 20 μL of peroxidase working reagent+10 μL of Trolox standard solution+170 μL of ABTS working reagent, blank group: 20 μL of peroxidase working reagent+10 μL of distilled water+170 μL of ABTS working reagent. The reaction system was gently mixed thoroughly and incubated in the dark at room temperature for 6 min. The absorbance was measured at 405 nm. The ABTS·+scavenging capacity was expressed as Trolox-equivalent antioxidant capacity (TEAC), in units of μmol TE/g.

(3) Determination of Superoxide Anion (O⁻₂) Scavenging Rate

The assay was performed referring to the instructions of the superoxide anion scavenging capacity assay kit. The working reagent was prepared immediately before use and peptide solutions at different concentrations were prepared. Experimental group: 20 μL of Working Xanthine Oxidase reagent+20 μL of peptide solution+80 μL of working reagent; control group: 20 μL of Working Xanthine Oxidase reagent+20 μL of distilled water+80 μL of working reagent; blank group: 40 μL distilled water+80 μL Working reagent. The reaction mixture was mixed thoroughly, and the absorbance A₀ at 450 nm was read immediately. After incubation at room temperature in the dark for 60 min, the absorbance A₆₀ was then read at 450 nm. Calculation was performed according to ΔA=A₆₀−A₀ and the results were recorded as ΔA_{blank}, ΔA_{control} and ΔAₜₑₛₜ. Calculation was further performed according to ΔΔA_{control}=ΔA_{control}−ΔA_{blank}, and ΔΔAₑₓₚₑᵣᵢₘₑₙₜₐₗ=ΔAₑₓₚₑᵣᵢₘₑₙₜₐₗ−ΔA_{blank}. The superoxide anion (O⁻₂) scavenging rate was calculated according to Formula 2:

$${{O}^{-}}_{2} scavenging rate ( \% ) = ( ΔΔ {A}_{control} - ΔΔ {A}_{experimental} ) / ΔΔ {A}_{control} \times 100 \% \qquad ( 2 )$$

### (4) Determination of Hydroxyl Radical (·OH) Scavenging Rate

First, 0.2502 g of FeSO₄ was weighed and dissolved in 100 mL of ultrapure water to prepare a 9 mmol/L FeSO₄ solution; 0.1243 g of salicylic acid was weighed and dissolved in 100 mL of anhydrous ethanol to prepare a 9 mmol/L salicylic acid solution; 0.1 mL of 30% H₂O₂ was taken and diluted to 100 mL with ultrapure water. 1 mL of FeSO₄ solution, 1 mL of salicylic acid solution, 1 mL of H₂O₂ solution, and 1 mL of the sample solution were taken and allowed to react thoroughly in the dark for 30 min. The absorbance was measured at 510 nm and recorded as A1. Ultrapure water was used to replace the sample solution, and the resulting absorbance after reaction was measured and recorded as A0. Ultrapure water was used to replace the H₂O₂ solution, and the resulting absorbance after reaction was measured and recorded as A2. The hydroxyl radical (·OH) scavenging rate was calculated according to Formula 3:

$$OH scavenging rate = ( 1 - \frac{A 1 - A 2}{A 0} ) \times 100 \% \qquad ( 3 )$$

### (5) Fe³⁺ Reducing Power Determination

The Fe³⁺ reducing power was determined by referring to the instructions of the Total Antioxidant Capacity Assay Kit (FRAP method). The FRAP working reagent was prepared and incubated at 37° C.; and the working reagent was prepared immediately before use. The antioxidant capacity of the antioxidant was expressed as the relative ferrous salt total antioxidant capacity. Therefore, FeSO₄·7H₂O was subjected to serial dilution to generate the standard curve. Experimental group: 5 μL of peptide solution+180 μL of FRAP working reagent; standard curve group: 5 μL of FeSO₄·7H₂O standard solution+180 μL of FRAP working reagent, blank group: 5 μL of distilled water+180 μL of FRAP working reagent. The reaction system was gently mixed thoroughly and incubated at 37° C. for 5 min, and then A593 was measured. The Fe³⁺ reducing power was expressed as the FeSO₄·7H₂O-equivalent antioxidant capacity, in units of μmol FeSO₄·7H₂O eq/g.

### 2. Experimental Results

### (1) Results and Analysis of DPPH·Free Radical-Scavenging Activity

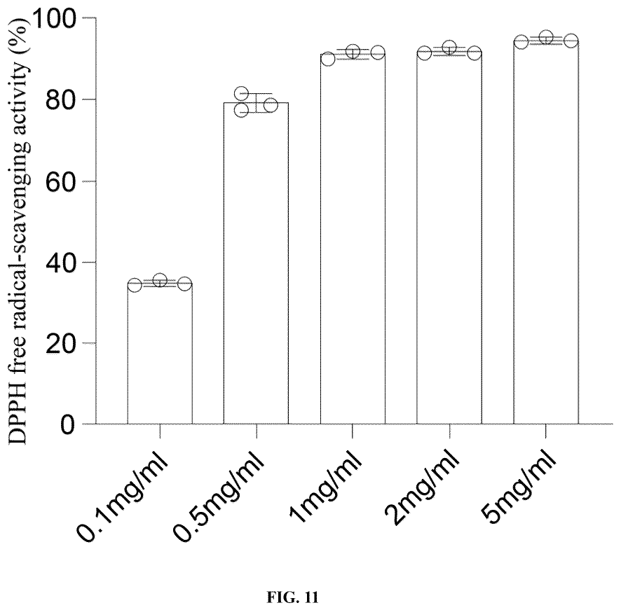

The DPPH·free radical-scavenging activity of the antioxidant peptide at different concentrations was shown in FIG. 11. It could be seen that the scavenging rate increased steadily with concentration, and exceeded 90% at 1 mg/mL, which was comparable to 1 mg/mL GSH. The results demonstrated that the peptide had strong DPPH·free radical-scavenging activity.

### (2) Results and Analysis of ABTS·+Scavenging Activity

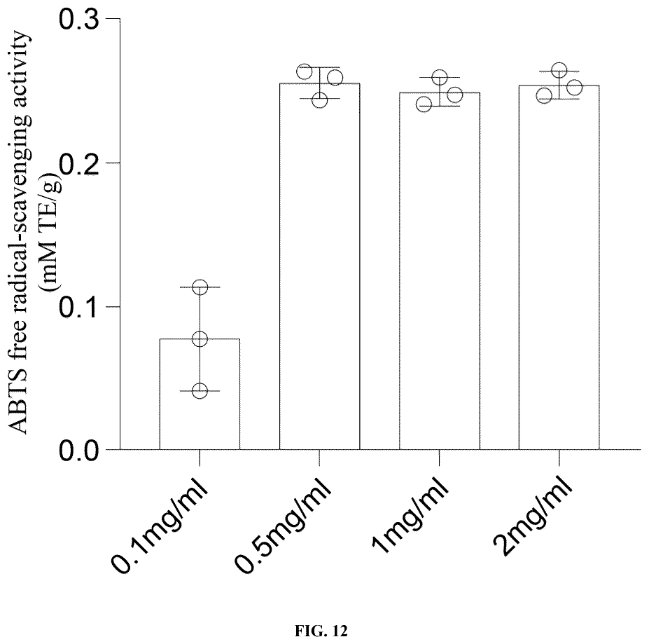

The ABTS·+free radical-scavenging activity of the antioxidant peptide at different concentrations was shown in FIG. 12. It could be seen that at 0.5 mg/mL, the antioxidant capacity was 0.255 mM TE/g. Thereafter, as the concentration increased, the antioxidant capacity remained stable within this range. The results demonstrated that the peptide had good and stable ABTS·+free radical-scavenging activity.

(3) Results and Analysis of Superoxide Anion (O⁻₂) Scavenging Activity

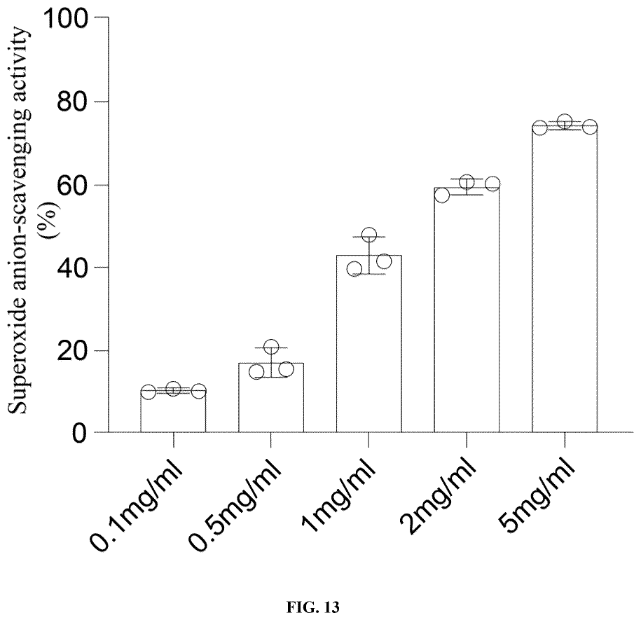

The superoxide anion (O⁻₂) scavenging activity of the antioxidant peptide at various concentrations was shown in FIG. 13. The O⁻₂ scavenging activity was strengthened with increasing polypeptide concentration, demonstrating a positive correlation. At 5 mg/mL, the scavenging rate reached 73.9%, indicating that the peptide has good O⁻₂ scavenging capacity.

### (4) Results and Analysis of Hydroxyl Radical (·OH) Scavenging Activity

The hydroxyl radical (·OH) scavenging activity of the antioxidant peptide was shown in Table 7 below. In this experiment, it was only detected that at 5 mg/mL, the hydroxyl radical (·OH) scavenging activity was 16.55%. At lower concentrations, the scavenging effect was poor. The results demonstrated that the peptide could possess hydroxyl radical (·OH) scavenging activity when the concentration increases.

TABLE 7
Hydroxyl radical (•OH) scavenging activity

| | | Hydroxyl | |
|---|---|---|---|
| | | radical | Average |
| Peptide | | scavenging | scavenging |
| sequences | Concentration | rate (%) | rate (%) |
| ALQPLPGR | 5 mg/mL | 15.32 | 16.55 |
| (SEQ ID | | 15.13 | |
| NO: 4) | | 19.20 | |

### (5) Results and Analysis of Fe³⁺ Reducing Power Determination

The Fe³⁺ reducing power of the antioxidant peptide was shown in Table 8 below, which was 35.79 μmol FeSO₄·7H₂O eq/g, indicating that the peptide has Fe³⁺ reducing power, but the reducing power was not as strong as the free radical scavenging effect.

TABLE 8
Fe³⁺ reducing power

| | | | | Average |
|---|---|---|---|---|
| | | | | reducing |
| | | | | power |
| | | Standard | Fe³⁺ | (μmol |
| Peptide | Concen- | curve | reducing | FeSO₄•7H₂O |
| sequences | tration | equation | power | eq/g) |
| ALQPLPGR | 2 mg/mL | Y = | 35.79 | 35.79 |
| (SEQ ID | | 0.2026x | 33.32 | |
| NO: 4) | | + 0.0465 | 38.25 | |

### Example 4: Protective Effects of Antioxidant Peptides from Mare's Milk Whey Protein on H₂O₂—Induced Oxidative Damage in HepG2 Cells

### 1. Experimental Steps and Methods

### (1) Cell Culture

Cell resuscitation: A cryovial containing HepG2 cells was removed from the liquid nitrogen tank and immediately placed in a 37° C. water bath for rapid thawing (within 1 min). 2 mL of culture medium was added to the vial, and the mixture was centrifuged at 1500 g for 5 min. The supernatant was discarded, and 2 mL of culture medium was added to resuspend the cells by gentle pipetting. The cell suspension was then transferred to a culture dish. The dish was tilted at a 30° angle and gently pipetted 10 times. The dish was placed in a 37° C. incubator with 5% CO₂ for continued culture.

Cell passage: the cells were passaged when the cell density reached 80-90% and the cells were in good condition. The original culture medium was aspirated, and the cells were washed by adding PBS gently along the wall of the dish. The PBS was aspirated, and 2 mL of 0.25% trypsin was added to the 10 cm culture dish for digestion for 3 min. Digestion was then terminated by adding 4 mL of serum-containing medium. The cells were detached by pipetting and collected into a 10 mL centrifuge tube. The cell suspension was centrifuged at 1500 g for 5 min, and the supernatant was discarded. The cells were resuspended in 3 mL of culture medium and passaged into a new culture dish. The dish was gently swirled to mix and left undisturbed for 15 min to allow cell sedimentation. Finally, the dish was placed in an incubator for continued culture.

### (2) CCK8 Cell Viability Assay

HepG2 cells were seeded in a 96-well plate at a density of 10⁴ cells/well. When the cell density reached 70-80%, the cells were treated with the corresponding drugs for a given period of time. After the drug treatment, 100 μL of culture medium containing 10% CCK8 reagent was added to each well. After incubation at 37° C. for 45 min, the absorbance was measured at 450 nm, and the cell viability was calculated according to Formula 4. Six technical replicates were set for each group.

$$Cell survival = ( {A}_{drug} - {A}_{blank} ) / ( {A}_{control} - {A}_{blank} ) \qquad ( 4 )$$

A_{drug}: containing cells, media, CCK-8 solution and drug solution
A_{blank}: cell-free drug-free media
A_{control}: containing cells, media, and CCK-8 solution, drug-free

### (3) Establishment of Oxidative Damage Model in HepG2 Cells

Upon reaching 80-90% confluence in the culture dish, the procedure followed the same steps as those prior to passaging and cryopreservation. The supernatant was discarded, and 1 mL or 2 mL of complete medium was added. The cell suspension was mixed thoroughly by pipetting. 10-20 μL of the cell suspension was taken and added to a hemocytometer for observation and counting under a microscope. The total cell count in the cell suspension was calculated, and the cells were diluted to a concentration of 1×10⁴ cells/mL. A 96-well cell culture plate was seeded with 100 μL of the diluted cell suspension per well and cultured in an incubator for 12-24 h until the cells were adherent and reached a moderate density. The supernatant was then discarded, and 100 μL of a series of H₂O₂ solutions (200, 400, 600, 800, 1000, 1200, 1400, and 1600 μM) prepared in complete medium was added. Six replicates were set for each concentration. The cells were cultured in an incubator for 12 h. Cell survival rate was determined using the CCK8 assay, and the H₂O₂ concentration resulting in approximately 50% cell survival was selected for subsequent use in the cell damage model.

### (4) Effect of the Antioxidant Peptide on Survival Rate of HepG2 Cells

Cells in the logarithmic growth phase were collected, and the subsequent procedure followed the same steps as in (3). The supernatant was discarded, and 1 mL or 2 mL of complete medium was added, followed by thorough mixing via pipetting. The cells were counted. The total cell count in the cell suspension was calculated, and the cells were diluted to a concentration of 1×10⁴ cells/mL. A 96-well cell culture plate was seeded with 100 μL of the diluted cell suspension and cultured in an incubator for 12-24 h until the cells were adherent and reached a moderate density. The supernatant was then discarded, and 100 μL of a series of peptide solutions (12.5, 25, 50, 100, 200, 400, 600, 800 μg/mL) prepared in complete medium was added. Six replicates were set for each concentration. The cells were cultured in an incubator for 24 h. The cell survival rate was determined using the CCK8 assay.

(5) Protective Effects of Antioxidant Peptides on H₂O₂-Induced Oxidative Damage in HepG2 Cells

Following the same procedure as in (3), the cells were seeded into a 96-well cell culture plate. After the cells reached confluence, the supernatant was discarded. Mixed solutions of serially diluted peptide solutions (prepared in complete medium at concentrations of 100 and 200 μg/mL) and 800 μM H₂O₂ were added, and the cells were cultured in an incubator for another 12 h. The supernatant was discarded and washed once with PBS, and the cell survival was determined using the CCK8 assay.

### (6) Determination of Cell-Related Antioxidant Indicators

Upon reaching 80-90% confluence, the procedure followed the same steps as those prior to passaging and cryopreservation. After resuspension, the cell suspension was diluted and seeded into a 6-well cell culture plate at 2 mL per well. The plate was then gently agitated to ensure even distribution and cultured in an incubator for 12-24 h. When the cells were in the logarithmic growth phase, the supernatant was discarded, and the cells were washed twice with PBS. Samples prepared in medium were then added. The control group, the sample group, and the model group were provided. For the control group, complete medium was added; for the model group, 800 μM H₂O₂ was added; and for the sample group, 200 μg/mL peptide solution+800 μM H₂O₂ were added. The cells were then incubated in an incubator for 12 h. The supernatant was discarded, and the cells were washed twice with PBS and digested with 500 μL of trypsin for 2 min. The digestion was terminated by adding 1 mL of medium. The cells were resuspended in PBS, transferred to 1.5 mL centrifuge tubes, and centrifuged at 1500 g for 5 min. The supernatant was discarded, 300 μL of PBS was added to the cell pellet and the mixture was kept on ice. The cells were subjected to ultrasonication for 1 min under the following conditions: 300 W power, with a cycle of 2 s on and 10 s off. The resulting supernatant of the disrupted cells was collected for the measurement of various indicators (Malondialdehyde (MDA), Superoxide Dismutase (SOD), Catalase (CAT), Glutathione Peroxidase (GSH-Px)). The activities of MDA, SOD, CAT, and GSH-Px were strictly determined in accordance with the steps outlined in the respective kit instructions. Proteins were quantified by the BCA assay.

### (7) Determination of Reactive Oxygen Species (ROS)

The initial cell culture, drug treatment, and induction steps were the same as those described in (6) for determining related antioxidant indicators. According to the ROS assay kit instructions, the DCFH-DA fluorescent probe was prepared in serum-free medium at a 1:1000 dilution for later use. After cell induction, the supernatant was discarded. Under light-protected conditions, 1 mL of the prepared 10 μM DCFH-DA fluorescent probe was added to the 12-well plate, and the plate was returned to the cell incubator and cultured for another 30 min. The plate was then removed (all subsequent steps were performed under light-protected conditions), and the cells were washed four times with PBS to thoroughly remove any excess background probe. Intracellular ROS fluorescence was observed using a confocal laser scanning microscope with excitation and emission wavelengths of 488 and 525 nm, respectively.

(8) Total Cellular RNA Extraction and qRT-PCR

The initial cell culture, modeling and drug treatment, and induction steps were the same as those described in (6) for determining related antioxidant indicators. After 12 h of modeling and drug treatment, the 6-well plate was taken out, the supernatant was discarded, and the cells were washed twice with PBS. 1 mL of Trizol was added to each well, and the cells were lysed on ice for 15 min. The contents in the six-well plate were repeatedly pipetted and collected into 1.5 mL RNase-free centrifuge tubes, respectively. 200 μL of chloroform (Trizol: chloroform=5:1) was added to each tube, followed by vigorous vortex mixing. The tubes were centrifuged at 4° C., 11000 g for 15 min. The supernatant was collected, and an equal volume of isopropanol was added. The tubes were inverted to mix and then placed in a −20° C. refrigerator for 2 h. The tubes were centrifuged at 4° C., 11000 g for 15 min. The supernatant was carefully discarded by inverting the tubes. The pellet was washed by adding 500 μL of 75% ethanol (prepared with 0.1% DEPC-treated water), vortexed briefly, and centrifuged at 4° C., 11000 g for 10 min. The supernatant was aspirated, leaving the pellet, i.e., the extracted RNA. The pellet was air-dried at room temperature and then dissolved in pre-warmed (60° C.) 0.1% DEPC-treated water. The RNA concentration was determined using a NanoDrop 2000. Reverse transcription was then performed using a cDNA synthesis kit, with the specific steps being carried out in accordance with the instructions. Following reverse transcription, real-time quantitative PCR (RT-qPCR) was performed using a fluorescent quantitative pre-mix kit and a real-time fluorescent PCR detection system. The reaction procedure and primer sequences (primer sequences were obtained from primer bank and validated by Blast) used were listed in Tables 9 and 10 below.

TABLE 9
RT-qPCR reaction procedure

| | | | | | Fluorescent |
|---|---|---|---|---|---|
| | Number | | | | signal |
| Stage | of cycles | Temperature | Time | Step | acquisition |
| Predenaturation | 1 | 95° C. | 60 s | Predenaturation | NO |
| | | 95° C. | 5 s | Denaturation | NO |
| PCR reaction | 40 | 58° C. | 10 s | Annealing | NO |
| | | 72° C. | 15 s | Elongation | YES |

TABLE 10
Primer sequences

| Genes | Forward primer | Reverse primer |
|---|---|---|
| | (5′-3′) | (5′-3′) |
| Keap1 | TGCCCCTGTGGTCAAAGTG | GGTTCGGTTACCGTCC |
| | (SEQ ID NO: 6) | TGC (SEQ ID NO: 7) |
| Nrf2 | TCTTGGAGTAAGTCGAGAA | GTTGAAACTGAGCGAAA |
| | GTGT (SEQ ID NO: 8) | AAGGC (SEQ ID NO: 9) |
| NQO1 | AGGATGGGAGGTACTCGAA | AGGCGTCCTTCCTTATAT |
| | TC (SEQ ID NO: 10) | GCTA (SEQ ID NO: 11) |
| SOD1 | AACCAGTTGTGTTGTCAGG | CCACCATGTTTCTTAGAGTG |
| | AC (SEQ ID NO: 12) | AGG (SEQ ID NO: 13) |
| β-actin | GGCTGTATTCCCCTCCATC | CCAGTTGGTAACAATGCCA |
| | G (SEQ ID NO: 14) | TGT (SEQ ID NO: 15) |

### 2. Experimental Results

(1) Establishment of H₂O₂-Induced Oxidative Damage Model in HepG2 Cells

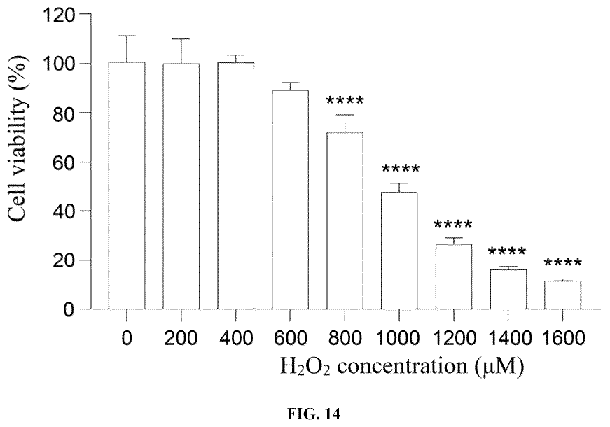

FIG. 14 showed the cell survival rate of HepG2 cells after 12 h of damage induced by H₂O₂ in the concentration range of 200-1600 μmol/L. As the H₂O₂ concentration increased, the survival rate of HepG2 cells decreased significantly (P<0.05). At 800 μmol/L, the survival rate was 71.97%, whereas at 1.0 mmol/mL, the mortality reached 47.72%. An H₂O₂ concentration of 800 μmol/L was selected to establish the subsequent cell damage model, since a lower survival rate might result in irreversible damage to the cells.

### (2) Effect of the Antioxidant Peptide on Survival Rate of HepG2 Cells

The effect of the antioxidant peptide on the survival rate of the HepG2 cells was shown in FIG. 15. At concentrations ranging from 12.5 to 800 μg/mL, the survival rate of the cells exceeded 100%, which suggests that the peptides were not only non-cytotoxic, but also had a protective effect on HepG2 cells. To avoid unnecessary consumption of polypeptides, concentrations of 100 and 200 μg/mL were selected for subsequent experiments.

(3) Protective Effects of Antioxidant Peptides on H₂O₂—Induced Oxidative Damage in HepG2 Cells

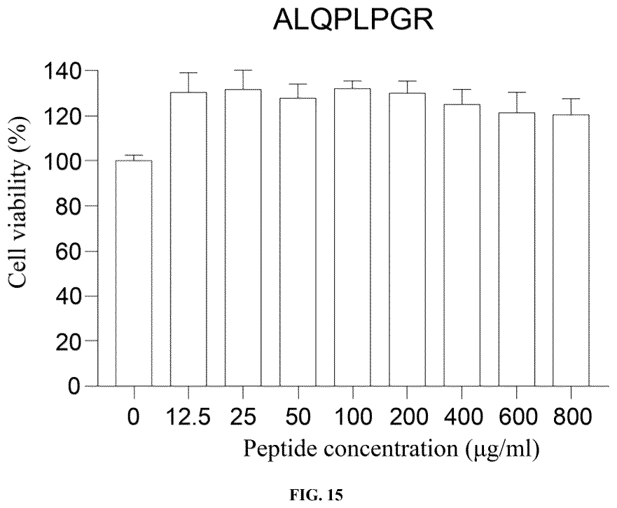

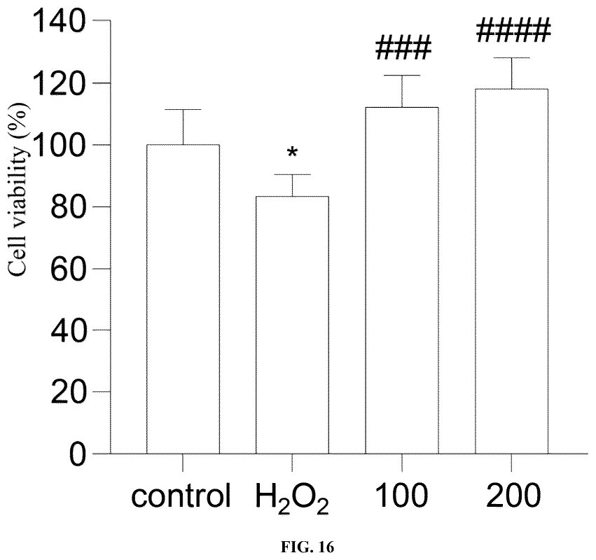

FIG. 16 showed the protective effect of the antioxidant peptide on HepG2 cells after 12 h of co-culture with H₂O₂. Compared with the model group, cells treated with antioxidant peptides at concentrations of 100 and 200 μg/mL showed a significantly higher survival rate. The 200 μg/mL concentration had a particularly significant effect. Therefore, the 200 μg/mL concentration was selected for further experiments.

### (4) Analysis of Relevant Antioxidant Enzyme Activities and Damage Markers

### a) Malondialdehyde (MDA)

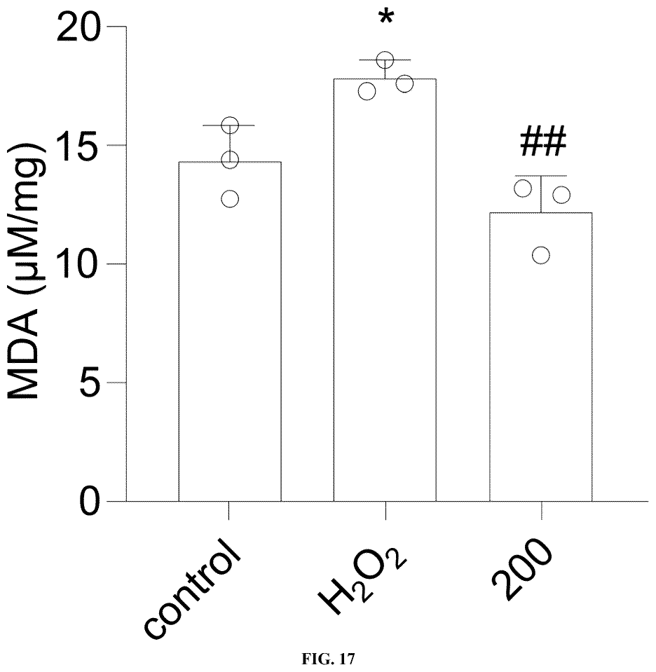

Malondialdehyde is a product of cellular lipid peroxidation, and a higher MDA content represents more severe oxidative stress damage. FIG. 17 showed that the cellular MDA content in the antioxidant peptide group was significantly reduced (12.14 μmol/mg) compared with the model group (17.81 μmol/mg).

### b) Total Superoxide Dismutase (T-SOD)

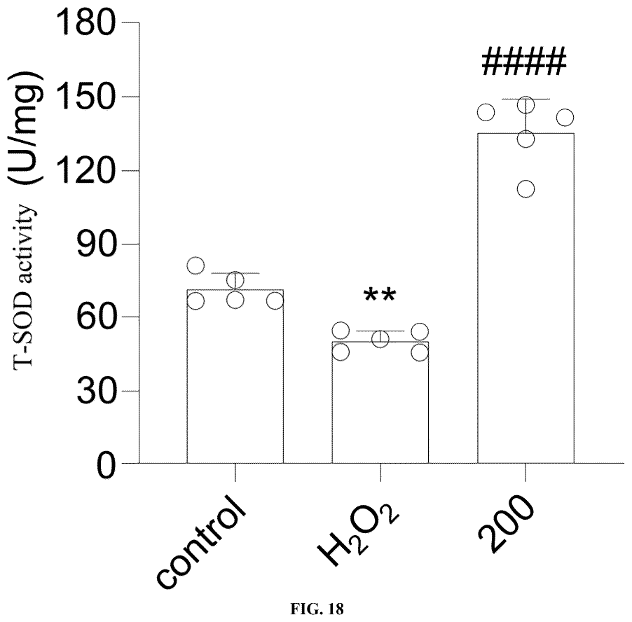

Total superoxide dismutase (T-SOD) is a critical antioxidant enzyme involved in the cellular defense against oxidative stress. FIG. 18 showed a significantly higher T-SOD enzyme activity in the antioxidant peptide-treated group (135.31 U/mg) compared with the model group (49.88 U/mg).

### c) Catalase (CAT)

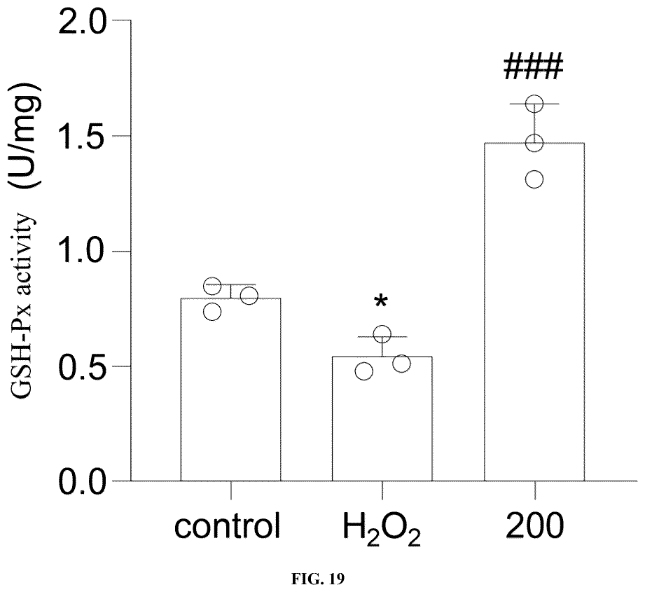

Catalase (CAT) can convert excessive hydrogen peroxide (H₂O₂) in cells into H₂O and O₂, thereby reducing oxidative stress and cellular damage. FIG. 19 showed that the cellular CAT enzyme activity in the antioxidant peptide group was significantly improved (2.95 U/mg) compared with the model group (2.26 U/mg).

### d) Glutathione Peroxidase (GSH-Px)

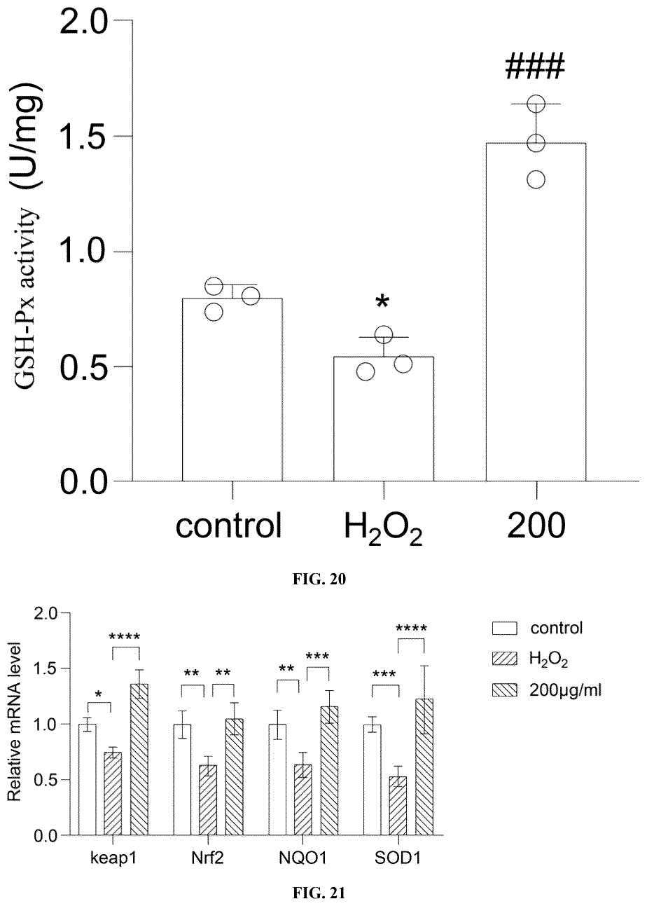

As shown in FIG. 20, the activity of the glutathione peroxidase (GSH-Px) in the antioxidant peptide group was significantly increased (1.47 U/mg) compared with the model group (0.54 U/mg).

### (5) Expression of Oxidative Stress Pathway-Related Genes

As the Keap1/Nrf2 signaling pathway plays a critical role in the occurrence and progression of oxidative stress, the expression of the genes related to this pathway was examined in this study. As shown in FIG. 21, the expression of Nrf2 and its downstream antioxidant genes HO-1, NQO1, and SOD1 was significantly decreased in the H₂O₂ group (P<0.01). In contrast, treatment with the antioxidant peptide significantly up-regulated the expression of these genes (P<0.05). These results suggest that the antioxidant peptide protects HepG2 cells from oxidative damage by regulating the Keap1/Nrf2 signaling pathway.

### (6) Reactive Oxygen Species (ROS)

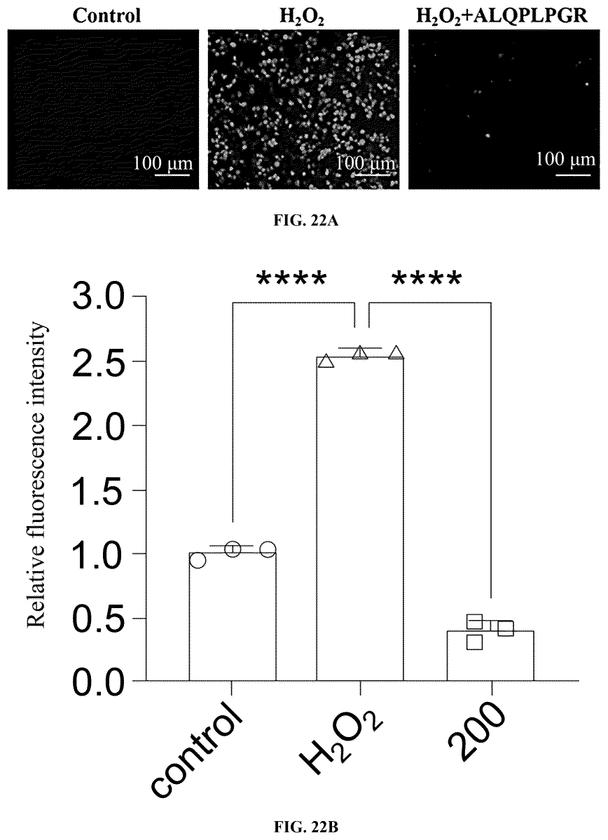

As clearly shown in FIG. 22A, the fluorescence intensity in the antioxidant peptide-treated group was significantly lower than that in the model group, indicating that the peptide reduces oxidative stress damage to the cells by scavenging intracellular ROS. As shown in the quantitative fluorescence diagram in FIG. 22B, the peptides significantly reduced the relative ROS content in cells compared with the model group (P<0.0001).

In summary, the antioxidant peptide ALQPLPGR (SEQ ID NO: 4) exhibited no cytotoxicity toward HepG2 cells and provided a protective effect against H₂O₂-induced damage in HepG2 cells. Analysis of cellular antioxidant indicators demonstrates that the antioxidant peptide modulated the balance of the antioxidant defense system in HepG2 cells.

The examples described above are only a part of, rather than all of the examples of the present disclosure. All other examples, obtained by those skilled in the art based on the examples of the present disclosure without creative efforts, shall fall within the scope of protection of the present disclosure.

What is claimed is:

1. An antioxidant peptide derived from mare's milk whey protein with antioxidant function, wherein the amino acid sequence of the antioxidant peptide derived from mare's milk whey protein is set forth in SEQ ID NO: 11, specifically: ALQPLPGR.

2. The antioxidant peptide derived from mare's milk whey protein according to claim 1, wherein the antioxidant peptide has free radical-scavenging activity.

3. Use of the antioxidant peptide according to claim 1 in preparation of food for defending against oxidative damage.

4. Use of the antioxidant peptide according to claim 2 in preparation of food for defending against oxidative damage.


SEQUENCE LISTING (15 sequences)

SEQ ID NO: 1 - protein, 181 aa, unidentified
MKCLLLALGLSLMCGNQATDIPQTMQDLDLQEVAGRWHSVAMVASDISLLDSESVPLRVYVEELRPTPEGNLEIILREGANHACVERNIVAQKTEDPAVFTVNYQGERKISVLDTDYAHYMFFCVGPPLPSAEHGMVCQYLARTQKVDEEVMEKFSRALQPLPGRVQIVQDPSGGQERCGF

SEQ ID NO: 2 - protein, 180 aa, unidentified
MKCLLLALGLALMCGIQATNIPQTMQDLDLQEVAGKWHSVAMAASDISLLDSESAPLRVYIEKLRPTPEDNLEIILREGENKGCAEKKIFAEKTESPAEFKINYLDEDTVFALDTDYKNYLFLCMKNAATPGQSLVCQYLARTQMVDEEIMEKFRRALQPLPGRVQIVPDLTRMAERCRI

SEQ ID NO: 3 - protein, 17 aa, unidentified
CVGPPLPSAEHGMVCQY

SEQ ID NO: 4 - protein, 8 aa, unidentified
ALQPLPGR

SEQ ID NO: 5 - protein, 7 aa, unidentified
LQPLPGR

SEQ ID NO: 6 - DNA, 19 bp, synthetic construct
  source 1..19: mol_type = other DNA
tgcccctgtggtcaaagtg

SEQ ID NO: 7 - DNA, 19 bp, synthetic construct
  source 1..19: mol_type = other DNA
ggttcggttaccgtcctgc

SEQ ID NO: 8 - DNA, 23 bp, synthetic construct
  source 1..23: mol_type = other DNA
tcttggagtaagtcgagaagtgt

SEQ ID NO: 9 - DNA, 22 bp, synthetic construct
  source 1..22: mol_type = other DNA
gttgaaactgagcgaaaaaggc

SEQ ID NO: 10 - DNA, 21 bp, synthetic construct
  source 1..21: mol_type = other DNA
aggatgggaggtactcgaatc

SEQ ID NO: 11 - DNA, 22 bp, synthetic construct
  source 1..22: mol_type = other DNA
aggcgtccttccttatatgcta

SEQ ID NO: 12 - DNA, 21 bp, synthetic construct
  source 1..21: mol_type = other DNA
aaccagttgtgttgtcaggac

SEQ ID NO: 13 - DNA, 23 bp, synthetic construct
  source 1..23: mol_type = other DNA
ccaccatgtttcttagagtgagg

SEQ ID NO: 14 - DNA, 20 bp, synthetic construct
  source 1..20: mol_type = other DNA
ggctgtattcccctccatcg

SEQ ID NO: 15 - DNA, 22 bp, synthetic construct
  source 1..22: mol_type = other DNA
ccagttggtaacaatgccatgt